(19) United States
(12) Patent Application Publication
(10) Pub. No.: US 2019/0374304 A1
(43) Pub. Date: Dec. 12, 2019

(54) DEVICE AND METHOD FOR INTERPROXIMAL REDUCTION

(71) Applicant: Brent K. Lopez, Denver, CO (US)

(72) Inventor: Brent K. Lopez, Denver, CO (US)

(21) Appl. No.: 16/434,753

(22) Filed: Jun. 7, 2019

Related U.S. Application Data
(60) Provisional application No. 62/681,787, filed on Jun. 7, 2018.

(51) Int. Cl. A61C 1/08

(52) U.S. Cl. CPC A61C 1/082; A61B 2034/105

(57) ABSTRACT

A guide for dental interproximal reduction, the guide including a tray shaped to substantially conform to one or more teeth, wherein the tray holds at least one of the one or more teeth stationary during the interproximal reduction; one or more grooves in the tray, each groove positioned to allow access to an interproximal space between a first tooth and a second tooth; and a first lateral edge in each of the one or more grooves, wherein the first lateral edge is shaped to guide an instrument for interproximal reduction of the first tooth.

### CROSS-REFERENCE TO RELATED APPLICATIONS

This patent application claims priority to U.S. provisional patent application Ser. No. 62/681,787 filed on Jun. 7, 2018 entitled “DEVICE AND METHOD FOR INTERPROXIMAL REDUCTION,” all of which is hereby incorporated by reference in its entirety.

### STATEMENT OF FEDERALLY FUNDED RESEARCH

Not applicable.

### TECHNICAL FIELD OF THE INVENTION

The present invention relates in general to the field of dental surgery. In particular, the present invention relates to interproximal reduction.

### BACKGROUND OF THE INVENTION

Without limiting the scope of the invention, its background is described in connection with orthodontic interproximal reduction.

One patent, U.S. Pat. No. 9,504,533, issued to Groscurth et al., is said to disclose a dental surgical drill guide assembly and method including a surgical guide housing and a base frame that fits to both gum tissue and one or multiple small areas of jawbone. The apparatus may be configured to accurately place dental implants according to the planned positions. Such apparatus may increase the stability of the surgical guide by clasping and/or contacting the jawbone, while improving the overall fit and minimizing the need of invasive surgery and flapping by also clasping and/or contacting selected area of gum tissue at the same time. Self-locking alignment members aid in easy removal, and re-installation, if the surgical guide housing to the base frame.

Another patent, U.S. Pat. No. 9,700,380, issued to Kim et al., is said to disclose a method for providing procedure guide for a dental implant, including: causing a computer tomography image and an oral scan image of a patient to acquire a combined three-dimensional image; selecting a fixture corresponding to a preset implant placement position, preparing the surgical guide in which at least one guide hole configured to guide drilling of a hole used to place the fixture is formed, and causing information about the placement of the fixture and information about drilling guidance to be calculated and prepared as information about a procedure report; outputting the procedure report; and delivering the output procedure report according to information about delivery of the surgical guide.

Another patent, U.S. Pat. No. 9,839,497, issued to Wouters, et al., is said to disclose and method of producing a surgical template comprising the steps of: obtaining a set of 3D surface data, the 3D surface data representing a surface of a patient's oral situation, obtaining a support structure model describing a surgical template configured to fit to a portion of the patient's oral situation, obtaining planned bore position data, the planned implant position data describing the longitudinal axes of a set of one or more planned implant bore holes relative to the 3D surface data, automatically modifying the support structure model to provide support material for guide holes in the support structure model corresponding to the one or more planned implant bore holes, producing the dental splint in dependence on the modified support structure model.

U.S. Patent Application Publication No. 2014/0205967, by Csapo et al., is said to disclose a method for dental restoration that includes disposing a flowable and curable tooth restoration composition into an open cavity of a stent that defines contours of a final design model of a patient's teeth and includes an interproximal contact that has been thinned along an interproximal contact zone but not separated, seating the stent on the patient's teeth such that the flowable and curable tooth restoration composition fills a space between at least one tooth to be restored and the stent, initiating curing of the flowable and curable tooth restoration composition in the stent such that the composition bonds to the at least one tooth to be restored and forms at least one restored tooth and removing the stent from the patient's teeth to expose the at least one restored tooth.

U.S. Patent Application Publication No. 2016/0278878, by Watson, et al., is said to disclose a sequential dental surgical guide system and method for immediate load is described. The system includes 1) a tooth and tissue borne surgical guide with fixation pins and bone reduction osteotomy sites 2) an implant osteotomy guide and 3) a prosthesis guide. The system does not rely on any anatomical reference points such as tissue or bone after placement of the first tooth and tissue borne reference guide. The sequential guide(s), both surgical implant placement and prosthesis guide rely on the original fixation positions determined by the first guide. Digital treatment planning and design are used to accurately place each sequential guide back to the original fixation pin positions. The fixation pin reference can be pinned and repined in the same sites per sequential guide or fixed in the jaw through the protocol by keeping them in place and switching out the guides.

In courses of orthodontic treatment, it is sometimes necessary to shape one or more teeth so that they may be aligned properly by removing small portions of outer surface material between teeth that touch or will touch when properly aligned, for instance when a jaw is slightly too small to accommodate full-sized teeth when properly aligned. When this is the case, interproximal reduction of one or more teeth is desirable. In an interproximal reduction, 0.3 mm or less of tooth enamel may be removed, requiring great precision in performing the procedure.

A practitioner performing an interproximal reduction or an interproximal dental enameloplasty may encounter a number of difficulties. These possible difficulties include a moving or mobile tooth, difficulty in reducing the correct amount of tooth, and inadvertently reducing a tooth such that a ledge or notch is created (“ledging”). When a patient is receiving orthodontic care, teeth are to a greater or lesser extent being moved very slowly, so that they are slightly loose or mobile and move when pressure, such as the pressure of an interproximal reduction instrument, is applied to them. Precision in interproximal reduction is difficult to achieve when this is the case. It is also difficult to remove the correct amount of tooth, given the small amounts at issue and the limitations inherent in manually manipulating an interproximal reduction instrument. Inadvertent creation of a ledge or notch is a specific difficulty of removing the correct amount, as when too much tooth is removed in a small part of the larger tooth portion to be reduced or when, while reducing one tooth, the interproximal reduction instrument touches the adjacent tooth across the interproximal space.

Existing devices and methods for interproximal reduction are inadequate to prevent or reduce the difficulties in performing the procedure. Devices for and methods of holding teeth stationary for interproximal reduction or an interproximal dental enameloplasty and guiding an interproximal reduction instrument or an interproximal dental enameloplasty instrument are desirable.

### SUMMARY OF THE INVENTION

In some embodiments of the disclosure, a guide for dental interproximal reduction is disclosed as including a tray shaped to substantially conform to one or more teeth, wherein the tray holds at least one of the one or more teeth stationary during the interproximal reduction; one or more grooves in the tray, each groove positioned to allow access to an interproximal space between a first tooth and a second tooth; and a first lateral edge in each of the one or more grooves, wherein the first lateral edge is shaped to guide an instrument for interproximal reduction of the first tooth. In one aspect, the first lateral edge includes a first stop to restrict a first range of the interproximal reduction to just above the gingival tissue. In another aspect, a second lateral edge in at least one of the one or more grooves is shaped to guide an instrument for interproximal reduction of the second tooth. In another aspect, the second lateral edge includes a second stop to restrict a second range of the interproximal reduction to just above the gingival tissue. In another aspect, the tray includes polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof. In another aspect, the instrument for interproximal reduction includes a reducing edge or surface including diamond, metal, silicates, or other materials that abrade tooth enamel.

In some embodiments of the disclosure, a set of guides for dental interproximal reduction is disclosed as including two or more trays, each tray configured for one in a sequence of interproximal reduction procedures in a course of orthodontic treatment of a patient, wherein each tray is shaped to substantially conform to one or more teeth, wherein the tray holds at least one of the one or more teeth stationary during the interproximal reduction, and each tray includes: one or more grooves in the tray, each groove positioned to allow access to an interproximal space between a first tooth and a second tooth; and a first lateral edge in each of the one or more grooves, wherein the first lateral edge is shaped to guide an instrument for interproximal reduction of the first tooth. In one aspect, the first lateral edge includes a first stop to restrict a first range of the interproximal reduction to just above the gingival tissue. In another aspect, a second lateral edge in at least one of the one or more grooves is shaped to guide an instrument for interproximal reduction of the second tooth. In another aspect, the second lateral edge includes a second stop to restrict a second range of the interproximal reduction to just above the gingival tissue. In another aspect, each tray includes polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof. In another aspect, the instrument for interproximal reduction includes a reducing edge or surface including diamond, metal, silicates, or other materials that abrade tooth enamel.

In some embodiments of the disclosure, kit for dental interproximal reduction is disclosed as including one or more trays, each tray configured for one in a sequence of interproximal reduction procedures in a course of orthodontic treatment of a patient, wherein each tray is shaped to substantially conform to one or more teeth, wherein the tray holds the at least one of the one or more teeth stationary during the interproximal reduction, and each tray includes: one or more grooves in the tray, each groove positioned to allow access to an interproximal space between a first tooth and a second tooth; and a first lateral edge in each of the one or more grooves, wherein the first lateral edge is shaped to guide an instrument for interproximal reduction of the first tooth; and one or more instruments for interproximal reduction. In one aspect, the first lateral edge includes a first stop to restrict a first range of the interproximal reduction to just above the gingival tissue. In another aspect, a second lateral edge in at least one of the one or more grooves is shaped to guide an instrument for interproximal reduction of the second tooth. In another aspect, the second lateral edge includes a second stop to restrict a second range of the interproximal reduction to just above the gingival tissue. In another aspect, each tray includes polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof. In another aspect, the instrument for interproximal reduction includes a reducing edge or surface including diamond, metal, silicates, or other materials that abrade tooth enamel.

In some embodiments of the disclosure, a method of guiding a dental interproximal reduction procedure is disclosed as including identifying a patient in need of the interproximal reduction procedure; providing at least one guide including: a tray shaped to substantially conform to one or more teeth, wherein the tray holds at least one of the one or more teeth stationary during the interproximal reduction; one or more grooves in the tray, each groove positioned to allow access to an interproximal space between a first tooth and a second tooth; and a first lateral edge in each of the one or more grooves, wherein the first lateral edge is shaped to guide an instrument for interproximal reduction of the first tooth;

providing an instrument for interproximal reduction; and using the at least one guide to perform the interproximal reduction procedure with the instrument for interproximal reduction. In one aspect, the first lateral edge includes a first stop to restrict a first range of the interproximal reduction to just above the gingival tissue. In another aspect, a second lateral edge in at least one of the one or more grooves is shaped to guide an instrument for interproximal reduction of the second tooth. In another aspect, the second lateral edge includes a second stop to restrict a second range of the interproximal reduction to just above the gingival tissue. In another aspect, each tray includes polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof. In another aspect, the instrument for interproximal reduction includes a reducing edge or surface including diamond, metal, silicates, or other materials that abrade tooth enamel.

In addition to the foregoing, various other method, system, and apparatus aspects are set forth in the teachings of the present disclosure, such as the claims, text, and drawings forming a part of the present disclosure.

The foregoing is a summary and thus contains, by necessity, simplifications, generalizations, and omissions of detail. Consequently, those skilled in the art will appreciate that this summary is illustrative only and is not intended to be in any way limiting. There aspects, features, and advantages of the devices, processes, and other subject matter described herein will be become apparent in the teachings set forth herein.

### BRIEF DESCRIPTION OF THE DRAWINGS

For a more complete understanding of the features and advantages of the present invention, reference is now made to the detailed description of the invention along with the accompanying figures, in which:

FIG. 1 depicts a guide for dental interproximal reduction.

FIG. 2 illustrates another view of the guide for dental interproximal reduction, with a groove for access to an interproximal space.

FIG. 3 shows the guide for dental interproximal reduction positioned to be applied to the teeth to which it is shaped to conform.

FIG. 4 shows the guide for dental interproximal reduction applied to the teeth.

FIG. 5 illustrates the guide for dental interproximal reduction applied to the teeth with a groove visible.

FIG. 6 depicts the guide for dental interproximal reduction with an instrument for interproximal reduction inserted into a groove.

FIG. 7 shows a flowchart of a method embodiment of the present invention.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

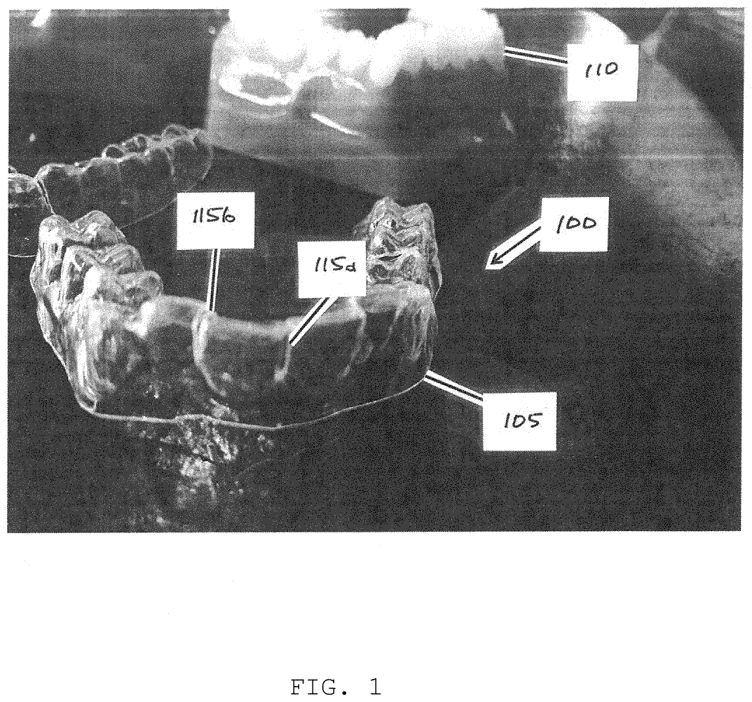

FIG. 1 depicts a guide for dental interproximal reduction. The guide 100 includes a tray 105 shaped to conform to the teeth 110 of a patient in need of interproximal reduction. The tray 105 is shaped to substantially conform to the teeth 110 of a patient needing interproximal reduction (herein, a patient's teeth are represented by a model). The tray 105 may be unitary tray or a tray in two or more separate segments. The tray 105 may be fabricated in a number of ways known to those skilled in the art, including 3D printing. The tray 105 holds the tooth or teeth requiring interproximal reduction stationary during the procedure. The tray 105 has one or more grooves positioned to allow access to the interproximal spaces in which the reduction or reductions are to be performed. Here, two such grooves 115a and 115b are shown. The guide may include plastic such as a polymer. Non-limiting examples of polymers or co-polymers for use with the present invention includes biocompatible materials including, but are not limited to, polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof. The tray 105 may be used on lower or upper teeth and is shaped to that it fits snuggly on a patient's teeth to keep them substantially stationary (“retention stabilization”) when pressures characteristic of interproximal reduction are applied to a tooth or teeth to be reduced.

Herein, the present invention is described in the context of interproximal reduction or an interproximal dental enameloplasty. Where only one of these procedures is discussed, one skilled in the art will recognize when the discussion is applicable to both procedures. The present invention is meant to eliminate operator error (such as “ledging” or performing a procedure on the wrong tooth); to provide protection by preventing gingival injury; to facilitate sequential, planned interproximal reduction during extensive orthodontic therapy, e.g., in cases of extreme crowding of teeth; to facilitate accuracy of a target enameloplasty; to provide stabilization/retention of the targeted tooth or teeth to shorten procedure time, in some cases drastically; and to greatly increase procedural efficiency.

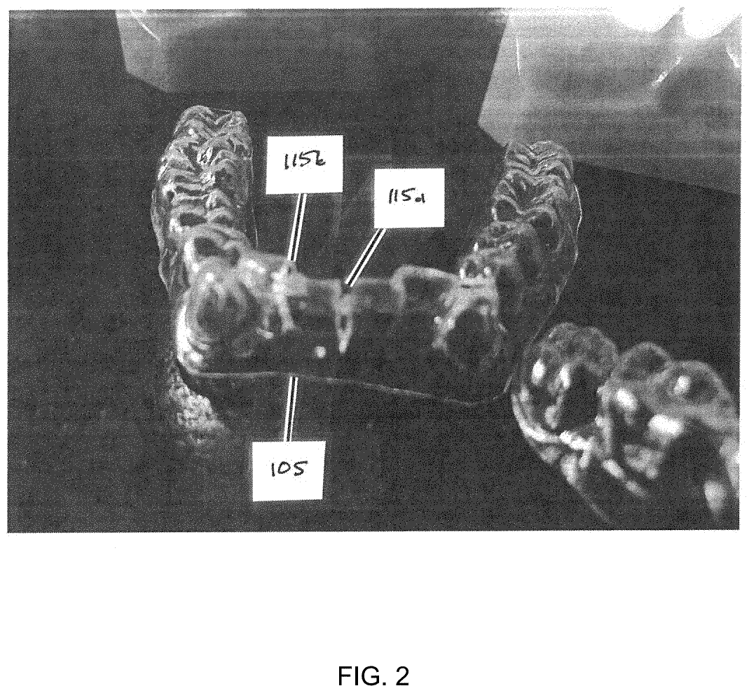

FIG. 2 illustrates another view of the tray 105, with a groove 115a for access to an interproximal space between a first tooth (not shown) and a second tooth (not shown) and a groove 115b for access to an interproximal space between a second tooth and a third tooth (not shown). Only two grooves are shown in FIG. 2, but the present invention encompasses guides with two or more such grooves, as well. Grooves 115a and 115b, and grooves in the tray 105 generally, may be closed at the bottom (the end of the groove toward the gingival tissue or “gums”) or open at the bottom, but if open, the bottom of the groove is far enough away from the gingival tissue to protect that tissue during the interproximal reduction or interproximal dental enameloplasty.

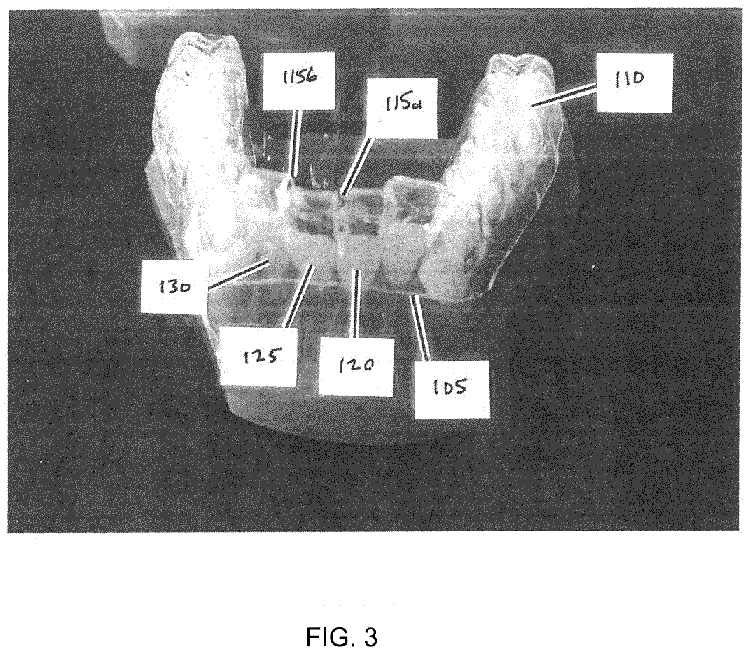

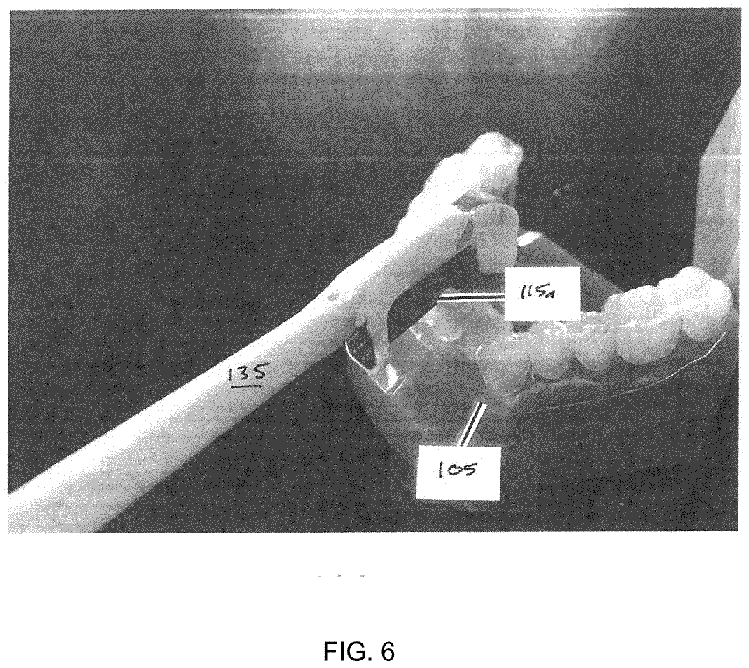

FIG. 3 shows the tray 105 positioned to be applied to the teeth 110 to which it is shaped to conform, with the grooves 115a and 115b shown. One or both of the lateral edges of grooves 115a and 115b are shaped to guide an instrument for interproximal reduction (see FIG. 6). The tray 105 will hold first tooth 120, second tooth 125, and third tooth 130 substantially stationary during the interproximal reductions. The practitioner performing the interproximal reductions may follow a shaped lateral edge of the grooves 115a and 115b to precisely reduce a tooth according to the planned alignment of the orthodontic course of treatment. One or both of the lateral edges of grooves 115a and 115b may also include a stop to restrict a range of an interproximal reduction to just above the gingival tissue or “guns” to prevent injury but below the interproximal contact area to allow for the necessary interproximal reduction or enameloplasty and to prevent ledging and operator error. One or both of the lateral edges of grooves 115a and 115b may be shaped to accommodate an interproximal reduction or an interproximal dental enameloplasty of one or both proximal surfaces at the same time, depending on the shape of the tooth or teeth to be which the procedure is to be applied under a therapy plan.

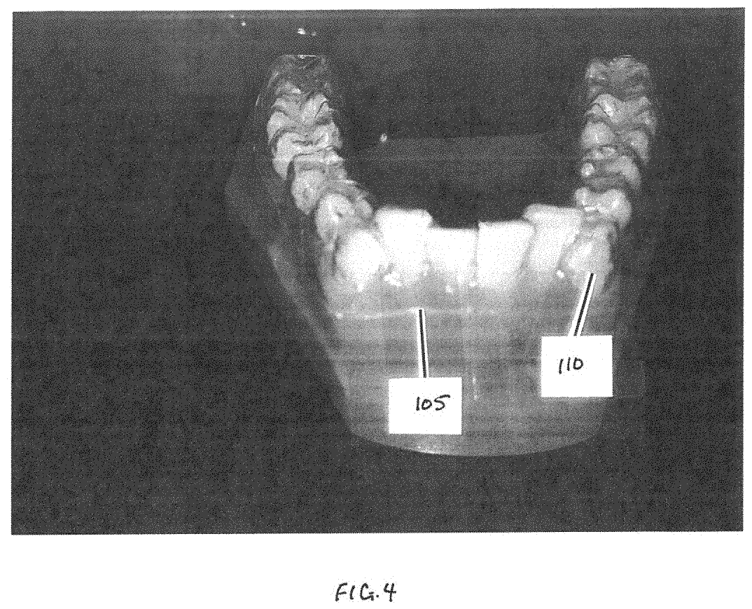

FIG. 4 shows the tray 105 applied to the teeth 110.

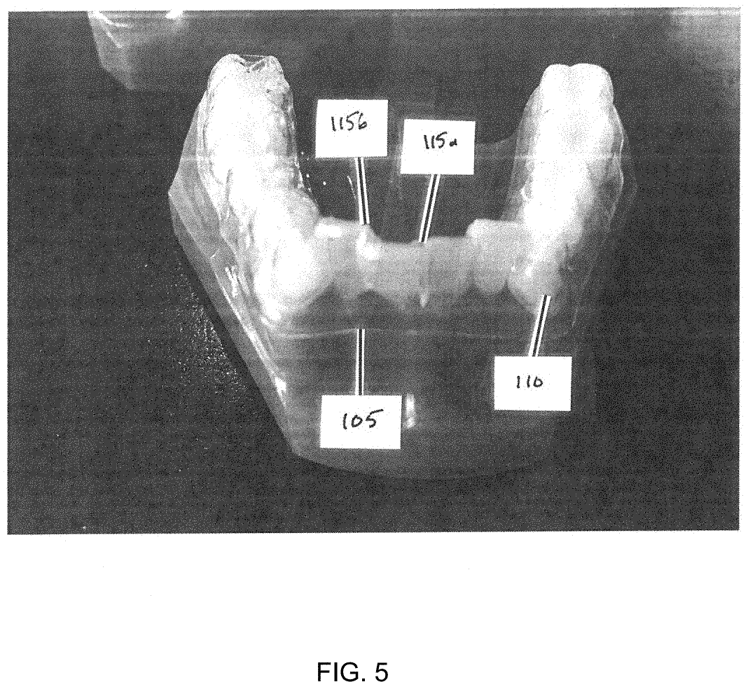

FIG. 5 illustrates the tray 105 applied to the teeth 110 with the grooves 115a and 115b visible.

FIG. 6 depicts the tray 105 with an instrument 135 for interproximal reduction inserted into the groove 115a. An instrument 135 for interproximal reduction may be a saw, a strip, or another implement well known to one skilled in the art of interproximal reduction. Instrument 135 may include a reducing edge or surface that includes diamond, metal, silicates, or other materials that abrade tooth enamel. Instrument 135 may include a single or double sided abrasive surface configured to accurately reduce or perform enameloplasty on one or both adjacent enamel surfaces, depending on the planned necessary enamel reduction.

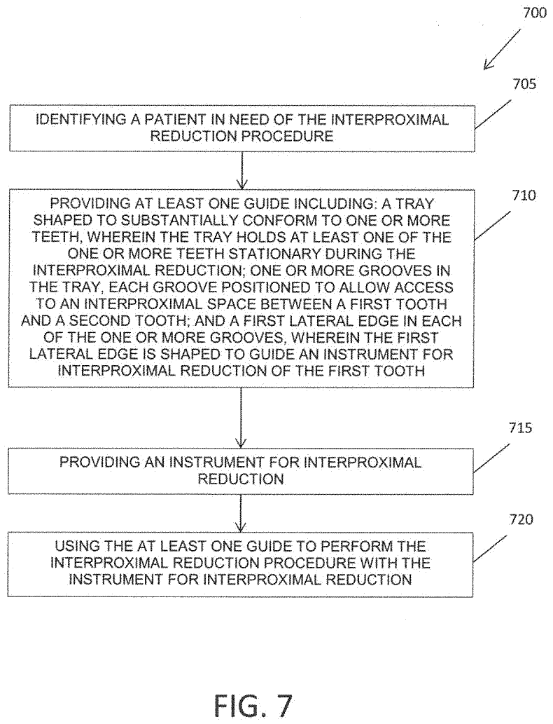

FIG. 7 shows a flowchart of a method embodiment of the present invention. Method 700 of guiding a dental interproximal reduction procedure begins with block 705, which includes identifying a patient in need of the interproximal reduction procedure. The method continues with providing at least one guide including a tray shaped to substantially conform to one or more teeth, wherein the tray holds at least one of the one or more teeth stationary during the interproximal reduction; one or more grooves in the tray, each groove positioned to allow access to an interproximal space between a first tooth and a second tooth; and a first lateral edge in each of the one or more grooves, wherein the first lateral edge is shaped to guide an instrument for interproximal reduction of the first tooth, found in block 710. Block 715 includes providing an instrument for interproximal reduction. Finally, at least one guide to perform the interproximal reduction procedure with the instrument for interproximal reduction is used in block 720.

Because a course of orthodontic treatment, including a sequence of interproximal reduction procedures, may be planned with accuracy for a long period, a set of guides 100, each guide 100 including a tray 105 with one or more grooves 115a, 115b, each groove 115a, 115b with one or two shaped lateral edges, may be prepared in advance for use by a practitioner or even by the dental patient. Such a set may be provided in a kit for interproximal reduction, and such a kit may include an interproximal reduction instrument.

The skilled artisan will recognize that the devices and methods described and claimed herein provide improvements in holding teeth stationary for interproximal reduction and guiding an interproximal reduction instrument.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

What is claimed is:

1. A guide for dental interproximal reduction, the guide comprising:
a tray shaped to substantially conform to one or more teeth, wherein the tray holds at least one of the one or more teeth stationary during the interproximal reduction;
one or more grooves in the tray, each groove positioned to allow access to an interproximal space between a first tooth and a second tooth; and
a first lateral edge in each of the one or more grooves, wherein the first lateral edge is shaped to guide an instrument for interproximal reduction of the first tooth.

2. The guide of claim 1, wherein the first lateral edge comprises a first stop to restrict a first range of the interproximal reduction to just above the gingival tissue.

3. The guide of claim 1, wherein a second lateral edge in at least one of the one or more grooves is shaped to guide an instrument for interproximal reduction of the second tooth; or
wherein the second lateral edge comprises a second stop to restrict a second range of the interproximal reduction to just above the gingival tissue.

4. The guide of claim 1, wherein the tray comprises polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof.

5. The guide of claim 1, wherein the instrument for interproximal reduction comprises a reducing edge or surface comprising diamond, metal, silicates, or other materials that abrade tooth enamel.

6. A set of guides for dental interproximal reduction, the set comprising:
two or more trays, each tray configured for one in a sequence of interproximal reduction procedures in a course of orthodontic treatment of a patient, wherein:
each tray is shaped to substantially conform to one or more teeth, wherein the tray holds at least one of the one or more teeth stationary during the interproximal reduction, and each tray comprises:
one or more grooves in the tray, each groove positioned to allow access to an interproximal space between a first tooth and a second tooth; and
a first lateral edge in each of the one or more grooves, wherein the first lateral edge is shaped to guide an instrument for interproximal reduction of the first tooth.

7. The set of guides of claim 6, wherein the first lateral edge comprises a first stop to restrict a first range of the interproximal reduction to just above the gingival tissue.

8. The set of guides of claim 6, wherein a second lateral edge in at least one of the one or more grooves is shaped to guide an instrument for interproximal reduction of the second tooth; or
wherein the second lateral edge comprises a second stop to restrict a second range of the interproximal reduction to just above the gingival tissue.

9. The set of guides of claim 8, wherein each tray comprises polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof.

10. The guide of claim 1, wherein the instrument for interproximal reduction comprises a reducing edge or surface comprising diamond, metal, silicates, or other materials that abrade tooth enamel.

11. A kit for dental interproximal reduction, the kit comprising:
one or more trays, each tray configured for one in a sequence of interproximal reduction procedures in a course of orthodontic treatment of a patient, wherein:
each tray is shaped to substantially conform to one or more teeth, wherein the tray holds the at least one of the one or more teeth stationary during the interproximal reduction, and each tray comprises:
one or more grooves in the tray, each groove positioned to allow access to an interproximal space between a first tooth and a second tooth; and
a first lateral edge in each of the one or more grooves, wherein the first lateral edge is shaped to guide an instrument for interproximal reduction of the first tooth; and
one or more instruments for interproximal reduction.

12. The kit of claim 11, wherein the first lateral edge comprises a first stop to restrict a first range of the interproximal reduction to just above the gingival tissue.

13. The kit of claim 11, wherein a second lateral edge in at least one of the one or more grooves is shaped to guide an instrument for interproximal reduction of the second tooth; or
wherein the second lateral edge comprises a second stop to restrict a second range of the interproximal reduction to just above the gingival tissue.

14. The kit of claim 11, wherein each tray comprises polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof.

15. The kit of claim 11, wherein the instrument for interproximal reduction comprises a reducing edge or surface comprising diamond, metal, silicates, or other materials that abrade tooth enamel.

16. A method of guiding a dental interproximal reduction procedure, the method comprising:
identifying a patient in need of the interproximal reduction procedure;
providing at least one guide comprising:
a tray shaped to substantially conform to one or more teeth, wherein the tray holds at least one of the one or more teeth stationary during the interproximal reduction;
one or more grooves in the tray, each groove positioned to allow access to an interproximal space between a first tooth and a second tooth; and
a first lateral edge in each of the one or more grooves, wherein the first lateral edge is shaped to guide an instrument for interproximal reduction of the first tooth;
providing an instrument for interproximal reduction; and
using the at least one guide to perform the interproximal reduction procedure with the instrument for interproximal reduction.

17. The method of claim 16, wherein the first lateral edge comprises a first stop to restrict a first range of the interproximal reduction to just above the gingival tissue.

18. The method of claim 16, wherein a second lateral edge in at least one of the one or more grooves is shaped to guide an instrument for interproximal reduction of the second tooth; or
wherein the second lateral edge comprises a second stop to restrict a second range of the interproximal reduction to just above the gingival tissue.

19. The method of claim 18, wherein the tray comprises polyetherurethane, polycarbonate, urethane, polyamide, polyimide, polyester, polypropylene, polyurethane silicone, polysiloxaneurethane, hydrogenated polystyrene-butadiene copolymer, ethylene-propylene and dicyclopentadiene terpolymer, and/or hydrogenated poly(styrene-butadiene) copolymer, poly(tetramethylene-ether glycol) urethanes, poly(hexamethylenecarbonate-ethylenecarbonate glycol) urethanes and combinations thereof.

20. The method of claim 18, wherein the instrument for interproximal reduction comprises a reducing edge or surface comprising diamond, metal, silicates, or other materials that abrade tooth enamel.
